(19) United States
(12) Patent Application Publication
(10) Pub. No.: US 2013/0123744 A1
(43) Pub. Date: May 16, 2013

(54) METHOD AND APPARATUS FOR THE DELIVERY OF POLYNUCLEOTIDE CANCER VACCINES TO MAMMALIAN SKIN

(71) Applicants: Derin C. Walters, Austin, TX (US); Alan D. King, Highland, MD (US); Anna-Karin Maltais (formerly Anna-Karin Roos), Spanga (SE); Britta Wahren, Djursholm (SE); Kristian Haller (formerly Kristian Hallermalm), Hasselby (SE); Andreas Brave, Stockholm (SE); Robert J. Walters, Severna Park, MD (US)

(72) Inventors: Richard E. Walters, Severna Park, MD (US); Derin C. Walters, Austin, TX (US); Alan D. King, Highland, MD (US); Anna-Karin Maltais (formerly Anna-Karin Roos), Spanga (SE); Britta Wahren, Djursholm (SE); Kristian Haller (formerly Kristian Hallermalm), Hasselby (SE); Andreas Brave, Stockholm (SE)

(21) Appl. No.: 13/138,722

(22) PCT Filed: Mar. 26, 2010
(86) PCT No.: PCT/US10/00908

§ 371 Date: Jan. 8, 2013

Related U.S. Application Data
(60) Provisional application No. 61/211,305, filed on Mar. 26, 2009.

(51) Int. Cl. A61N 1/30

(52) U.S. Cl. CPC A61N 1/306
USPC 604/501

(57) ABSTRACT

The invention provides a method and apparatus for the delivery of polynucleotide cancer vaccines to increase T cell response and reduce pain due to long electric waveform application and muscle contractions. The method includes the steps of: (a.) administering a polynucleotide cancer vaccine into the skin at an administration site, (b.) applying a needle electrode to the skin in the vicinity of the administration site, and (c.) applying a sequence of at least three electrical waveforms to deliver the polynucleotide cancer vaccine into skin cells by electroporation. The sequence has at least one of the following characteristics {1} at least two of the waveforms differ in amplitude, {2} at least two of the waveforms differ in width, and (3) a first waveform interval for a first set of two of the waveforms is different from a second waveform interval for a second set of two of the waveforms.

### CROSS-REFERENCE TO RELATED APPLICATION

This application claims priority based upon copending U.S. Provisional Application Ser. No. 61/211,305, filed 26 Mar. 2009, entitled “METHOD AND APPARATUS FOR THE DELIVERY OF POLYNUCLEOTIDE CANCER VACCINES TO MAMMALIAN SKIN”, by inventors Richard Walters (Columbia, Md.), Derin Walters (Tokyo, Japan), Alan King (Highland, Md.), Anna-Karin Roos (Stockholm, Sweden), Britta Wahren (Stockholm, Sweden), Kristian Hallermalm (Stockholm, Sweden), and Andreas Brave (Stockholm, Sweden).

### TECHNICAL FIELD

The present invention relates generally to methods and apparatus for the delivery of polynucleotide cancer vaccines to mammalian skin. More specifically, the present invention provides methods and apparatus for the delivery of polynucleotide vaccines to mammalian skin using electrical waveforms and electroporation.

### BACKGROUND ART

For purposes of the present disclosure, the term “pulse interval” means the time from the beginning of one pulse to the beginning of the next pulse.

The following publications are discussed hereinbelow:
U.S. Pat. No. 6,010,613;
U.S. Pat. No. 6,603,998;
U.S. Pat. No. 6,713,291;

“Enhancement of Cellular Immune Response to a Prostate Cancer DNA Vaccine by Intradermal Electroporation”, by Roos et al, Molecular Therapy, Vol. 13, No. 2, February 2006, pages 320-327 (referred to herein as Roos et al); and

“The effect of pulse repetition frequency on the uptake into electropermeabilized cells in vitro with possible applications in electrochemotherapy”, by Pucihar et al, Bioelectrochemistry 57 (2002) pages 167-172 (referred to herein as Pucihar et al).

U.S. Pat. No. 6,010,613, incorporated herein by reference, discloses using electroporation with wide interval electrical waveforms, such as provided by PA-4000 System (referred to herein as PulseAgile) of Cyto Pulse, Inc., 810 Cromwell Park Drive, Suite T, Glen Burnie, M D 21061. More specifically, U.S. Pat. No. 6,010,613 discloses applying a sequence of at least three single, operator-controlled, independently programmed, DC electrical pulses, to a material, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses.

For purposes of the discussions and disclosures herein, the above-mentioned applying a sequence of at least three single, operator-controlled, independently programmed, DC electrical pulses, to a material, with the characteristics (1), (2), and (3) set forth is referred to herein as “PulseAgile”.

The specification disclosed in U.S. Pat. No. 6,010,613 and the documentation connected with the PulseAgile system provide that the pulse interval is equal to or greater than 0.1 seconds, which is 100 milliseconds. Hereinafter, the PulseAgile generated electrical waveforms which have pulse intervals which are equal to or greater than 100 milliseconds are referred to as “wide interval PulseAgile electrical waveforms” or “slow PulseAgile electrical waveforms”.

In U.S. Pat. No. 6,010,613, there is no specific evidence presented that administered vaccines have either successful genetic expression of the vaccine or provide improved T-cell response involving improved secretion of good protein resulting from successful genetic expression of the vaccine.

Both U.S. Pat. No. 6,603,998 and U.S. Pat. No. 6,713,291, both incorporated herein by reference, disclose the delivery of polynucleotide vaccines to biological cells using the wide interval PulseAgile electrical waveforms or slow PulseAgile electrical waveforms.

Roos et al disclose the use of the wide interval PulseAgile electrical waveforms or slow PulseAgile electrical waveforms to deliver a polynucleotide vaccine to mammalian skin cells. It is also disclosed by Roos et al that successful genetic expression of the polynucleotide vaccine is demonstrated by detection of a genetic marker which expresses luciferase protein. In addition, Roos et al disclose that with the use of the wide interval PulseAgile electrical waveforms or the slow PulseAgile electrical waveforms to deliver a polynucleotide vaccine to mammalian skin cells, there is improved T-cell response involving improved secretion of good protein resulting from successful genetic expression of the polynucleotide vaccine. In Roos et al, T-cell response is represented by PSA-specific IFN(gamma)-producing CD8+ T cells.

Aside from the beneficial results disclosed in the Roos et al publication, there are two undesirable results observed by using the slow PulseAgile electrical waveforms. The first undesirable result is that each slow PulseAgile electrical waveform administration protocol took approximately 3.5 seconds. Since administration employing the use of needles penetrating into mammalian skin causes discomfort or pain, for such 3.5 second administration protocol, the mammal would have to endure the discomfort or pain for approximately 3.5 seconds.

The second undesirable result disclosed in Roos et al is that each slow PulseAgile electrical waveform causes a perceptible muscle contraction. The muscle contraction itself can also cause discomfort or pain. Normally, for an administration of a polynucleotide vaccine, plural pulsed waveforms would be applied to a mammal. Therefore, plural muscle contractions, with plural additional muscle discomfort or pain, would take place with such slow PulseAgile electrical waveforms.

Pucihar et al disclose that, before their publication date in 2002, electrical pulses have been used in combination with chemotherapeutic agents to treat cancerous cells. The earlier electrical pulses have had a frequency of 1 Hz, whereby each pulse produced a related tetanic contraction (muscle contraction). It is noted that 1 Hz translates to 1000 milliseconds per cycle. The discussed electrical pulse protocols are all pulse sequences that have pulses of uniform pulse amplitude, uniform pulse width, and uniform pulse interval. The chemotherapeutic agents include small nonpermeant hydrophilic molecules. The disclosures of the research conducted by Pucihar et al relate to in vitro (not in vivo) experiments with cancerous cell being treated with Lucifer Yellow, which is a small nonpermeant hydrophilic molecule. The disclosures of the research conducted by Pucihar et al explore various pulse repetition frequencies in order to exceed the frequency of tetanic contraction (so that successive muscle contractions fuse into smooth motion). There is a statement in Pucihar et al that with a frequency of excitation of 40 Hz, successive muscle contractions fuse into smooth motion. The 40 Hz pulse frequency employs pulses of uniform pulse amplitude, uniform pulse width, and uniform pulse interval. It is noted that 40 Hz translates to 25 milliseconds per cycle.

There is no disclosure by Pucihar et al that of any of their findings have any relationship to polynucleotide vaccination, to successful genetic expression of a polynucleotide vaccine, or to improved T-cell response involving improved secretion of a desired protein resulting from successful genetic expression of the polynucleotide vaccine.

Carcinoembryonic antigen (CEA) is a highly glycosylated membrane protein with a molecular weight of about 180 kDa. It is expressed during the fetal period, particularly in the fetal gut. After birth, CEA expression is down regulated, but persists in low amounts in the adult gut. CEA is over expressed by a large number of epithelial neoplasias, including colorectal cancer cells, and to a lesser degree in gastric, pancreatic, breast, lung and ovarian carcinomas (Wahren and Harmenberg 1991; Hammarstrom 1999). CEA is homogeneously expressed on the cell surface of more than 90% of colorectal carcinomas. It is not expressed in other cells of the body, except for fetal digestive organs and a low-level expression on normal colonic mucosa. The favorable expression profile and the possible role of CEA in tumorigenesis makes this antigen an attractive target for immunotherapy (Berinstein 2002). CEA is measurable in serum and widely used as a serologic marker for malignancy, especially in colorectal carcinoma.

The CEA subgroup members are cell membrane associated and show a complex expression pattern in normal and cancerous tissues with notably CEA showing a selective epithelial expression. Several CEA subgroup members possess cell adhesion properties and the primordial member, biliary glycoprotein, seems to function in signal transduction or regulation of signal transduction possibly in association with other CEA sub-family members (Hammarstrom 1999).

### Immunogenicity of CEA

There is evidence that the immune system can recognize CEA. Moreover, the presence of naturally occurring anti-CEA IgM antibodies has been associated with improved survival of patients with colorectal cancer (Albanopoulos et al. 2000). T cells from healthy donors and patients can recognize processed epitopes of CEA and lyse CEA-expressing tumors in the context of multiple HLA class I alleles (Berinstein 2002). Functional HLA-DR epitopes of CEA in patients have also been defined (Ullenhag et al. 2004).

Vaccination of Cancer Patients Targeting CEA

Numerous clinical trials targeting CEA have demonstrated that tolerance to CEA can be reversed. Anti-CEA immune responses are reproducibly generated by different vaccination approaches (Berinstein 2002; Marshall 2003). Clinical responses and survival improvement of vaccinated patients have also been reported.

Preliminary analysis of a Phase III study using an anti-idiotypic antibody mimicking CEA (CeaVac) demonstrated a trend toward overall survival improvement in patients with metastatic colorectal carcinoma receiving at least 5 doses of CeaVac versus placebo (Foon et al. 1999; Titan Pharmaceuticals 2002). CEA protein produced in a baculovirus expression system and conjugated to alum in combination with the adjuvant GM-CSF induced CEA-specific humoral and proliferative T-cell responses in colorectal carcinoma patients in the adjuvant setting. A positive correlation between anti-CEA IgG titers and overall survival of patients was demonstrated (Ullenhag et al. 2002; Ullenhag et al. 2004).

DC-based vaccinations targeting CEA have shown clinical responses and induced CEA-specific CD4+ helper T-cell and CD8+ cytotoxic T-cell responses (Morse et al. 2003; Liu et al. 2004; Matsuda et al. 2004; Ueda et al. 2004). Fong et al. reported dramatic tumor regression in 17% of immunized patients. In addition, 25% of patients had mixed response or stables disease. A strong correlation was demonstrated between the clinical response and the percentage as well as the magnitude of CEA-specific CD8+ effector T cells (Fong et al. 2001). Although DC-based vaccinations induce powerful immunity, several factors limit the use of this methodology in large-scale clinical trials (Berzofsky et al. 2004).

Genetic immunization offers several advantages over cell-based and protein vaccines (Liu 2003). Ease of manipulation, flexibility of design, greater chemical stability, the inherent adjuvant effect of unmethylated CpG oligodeoxynucleotides and low cost make DNA attractive for vaccination. DNA vaccines facilitate CTL induction, viewed to be crucial for anti-tumor defense.

Vaccination with DNA alone or recombinant vaccinia virus encoding CEA have not shown any objective clinical responses and the induced CEA-specific immune responses have been limited (Tsang et al. 1995; Conry et al. 1999; Conry et al. 2000; Conry et al. 2002). However, a prime-boost approach using vaccinia-CEA and another poxvirus vector, ALVAC containing CEA resulted in increased CEA-specific CTL precursor frequency and disease stabilization for up to 21 months in patients with advanced colorectal cancer, demonstrating that vaccination with CEA can be beneficial to the patient. CEA-specific IFN-γ response was associated with improved overall survival. GM-CSF significantly augmented the cellular response (Marshall et al. 2000; Marshall 2003). CTL induction and stable diseases were also seen in patients immunized with ALVAC-CEA containing the co-stimulatory molecule, B7.1 (Horig et al. 2000; von Mehren et al. 2000). Stable disease was more frequently induced in patients receiving GM-CSF (von Mehren et al. 2001).

Prime-boost vaccination with fowlpox-CEA-TRICOM and vaccinia-CEA-TRICOM containing the transgene for CEA and a triad of co-stimulatory molecules (B7.1, ICAM-1 and LFA-3) was associated with only one complete response. However, several patients having progressive disease at study entry had stable disease for more than 6 months after vaccination. Tumor marker responses were observed in some patients (Marshall et al. 2005). Vaccination with dendritic cells modified with fowlpox-CEA-TRICOM induced a higher CEA-specific CTL precursor frequency in patients with a minor clinical response or stable disease as compared to those who progressed.

Thus, although against common vaccine principles, it is made likely by us that tolerance to the endogenously produced CEA may in fact be broken and the immune system made to target tumor cells expressing CEA.

Safety of Vaccines Targeting CEA in Humans

Neither serious adverse events nor autoimmune toxicity was observed in patients using any of the vaccination strategies targeting CEA.

DNA vaccine encoding CEA and hepatitis B surface antigen have been used in a dose-escalation clinical trial in patients with colorectal cancer (Conry et al. 2002). Toxicity was limited to transient grade 1 local or systemic adverse events and was not dose-related.

Except for one grade II local injection site reaction, only grade I systemic or local adverse events were reported in patients vaccinated with CEA protein in conjunction with GM-CSF (Ullenhag et al. 2004). No evidence of autoimmune toxicity was demonstrated after a prolonged observation period (6 years).

Immunization with different prime-boost regimens targeting CEA were also safe and GM-CSF had no added toxicity (Berinstein 2002; Marshall 2003; Marshall et al. 2005)

Additional References with respect to CEA:
1. Albanopoulos, K, et al. (2000). Am J Gastroenterol 95(4): 1056-61
2. Beauchemin N, Benchimol S, Cournoyer D, Fuks A, Stanners C P. Isolation and characterization of full-length functional cDNA clones for human carcinoembryonic antigen. Mol Cell Biol. 1987 September; 7(9):3221-30
3. Berinstein, N L (2002). J Clin Oncol 20(8): 2197-207.
4. Berzofsky, J A, et al. (2004). J Clin Invest 113(11): 1515-25.
5. Collins J J, Black P H. Specificity of the carcinoembryonic antigen (CEA). N Engl J. Med. 1971 Jul. 15; 285(3):175-6.
6. Conry, R M, et al. (2000). Clin Cancer Res 6(1): 34-41.
7. Conry, R M, et al. (2002). Clin Cancer Res 8(9): 2782-7.
8. Conry, R M, et al. (1999). Clin Cancer Res 5(9): 2330-7.
9. Fong, L, et al. (2001). Proc Natl Acaci Sci USA 98(15): 8809-14.
10. Foon, K A, et al. (1999). J Clin Oncol 17(9): 2889-5.
11. Galaktionov V G, Evolutionary Development of the immunoglobulins super family, Izv Akad Nauk Ser Biol, 2004 March-April; (2):133-45 (in Russian; English trans. of abstract, see NLM Gateway)
12. Hallermalm, K, et al. (2007). Scand J Immunol 66(1): 43-51.
13. Hammarstrom, S (1999). Semin Cancer Biol 9(2): 67-81.
14. Horig, H, et al. (2000). Cancer Immunol Immunother 49(9): 504-14.
15. Liu, K J, et al. (2004). Clin Cancer Res 10(8): 2645-51.
16. Liu, M A (2003). J Intern Med 253(4): 402-10.
17. Lund, L H, et al. (2003). Cancer Gene Ther 10(5): 365-76.
18. Marshall, J (2003). Semin Oncol 30(3 Suppl 8): 30-6.
19. Marshall, J L, et al. (2005). J Clin Oncol 23(4): 720-731.
20. Marshall, J L, et al. (2000). J Clin Oncol 18(23): 3964-73.
21. Matsuda, K, et al. (2004). Cancer Immunol Immunother 53(7): 609-16.
22. Morse, M A, et al. (2003). Cancer Invest 21(3): 341-9.
23. Paxton R J, Mooser G, Pande H, Lee T D, Shively J E, Sequence analysis of carcinoembryonic antigen: identification of glycosylation sites and homology with the immunoglobulin supergene family. Proc Natl Acad Sci USA. 1987 February; 84(4):920-4
24. Titan Pharmaceuticals, I. (2002). “http://www.titanpharm.com/press/CeaVac_PhaseIII_Results.html.”
25. Tsang, K Y, et al. (1995). J Natl Cancer Inst 87(13): 982-90.
26. Ueda, Y, et al. (2004). Int J Oncol 24(4): 909-17.
27. Ullenhag, G J, et al. (2004). Cancer Immunol Immunother 53(4): 331-7.
28. Ullenhag, G J, et al. (2004). Clin Cancer Res 10(10): 3273-81.
29. Ullenhag, G J, et al. (2002). Cancer Res 62(5): 1364-9.
30. Wahren, B and U Harmenberg (1991). Scand J Clin Lab Invest Suppl 206: 21-7.
31. von Mehren, M, et al. (2001). Clin Cancer Res 7(5): 1181-91.
32. von Mehren, M, et al. (2000). Clin Cancer Res 6(6): 2219-28.
33. Zimmermann W, Ortlieb B, Friedrich R, von Kleist S. Isolation and characterization of cDNA clones encoding the human carcinoembryonic antigen reveal a highly conserved repeating structure. Proc Natl. Acad Sci USA. 1987 May; 84(9):2960-4.
34. U.S. Pat. No. 7,279,464 DNA vaccines encoding CEA and a CD40 ligand and methods of use thereof.

In view of the above, it would be desirable to provide a method and apparatus for the delivery of polynucleotide vaccine to mammalian skin which takes less than 3.5 seconds to administer the polynucleotide vaccine.

In addition, it would be desirable to provide a method and apparatus for the delivery of polynucleotide vaccines to mammalian skin which applies plural PulseAgile electrical waveforms to the mammalian skin and only causes one muscle contraction for the plural applied electrical waveforms.

Administration of a polynucleotide vaccine, to be successful, must give evidence of successful genetic expression of the administered polynucleotide vaccine. Moreover, to be successful, the genetic expression of the administered polynucleotide must give evidence of providing a desired protein which results from the successful genetic expression of the polynucleotide vaccine.

Thus, while the foregoing body of prior art indicates it to be well known to use electroporation apparatuses, the prior art described above does not teach or suggest a method and apparatus for the delivery of polynucleotide vaccines to mammalian skin which has the following combination of desirable features: (1) provides a method and apparatus for the delivery of polynucleotide vaccine to mammalian skin which takes less than 3.5 seconds to administer the polynucleotide vaccine; (2) applies plural PulseAgile electrical waveforms to the mammalian skin and only causes one muscle contraction for the plural applied electrical waveforms; (3) gives evidence of successful genetic expression of the administered polynucleotide vaccine; and (4) gives evidence of providing a desired protein which results from the successful genetic expression of the polynucleotide vaccine. The foregoing desired characteristics are provided by the unique method and apparatus for the delivery of polynucleotide vaccines to mammalian skin of the present invention as will be made apparent from the following description thereof. Other advantages of the present invention over the prior art also will be rendered evident.

### DISCLOSURE OF INVENTION

In accordance with one aspect of the invention, a method for the delivery of polynucleotide vaccines to mammalian skin includes the steps of:

(a.) administering a polynucleotide vaccine into the skin at an administration site,

(b.) applying a needle electrode to the skin in the vicinity to the administration site, and

(c.) applying a sequence of at least three single, operator-controlled, independently programmed, narrow interval electrical waveforms, which have pulse intervals that are less than 100 milliseconds, to deliver the polynucleotide vaccine into skin cells by electroporation. The sequence of at least three waveforms has one, two, or three of the following characteristics (1) at least two of the at least three waveforms differ from each other in waveform amplitude, (2) at least two of the at least three waveforms differ from each other in waveform width, and (3) a first waveform interval for a first set of two of the at least three waveforms is different from a second waveform interval for a second set of two of the at least three waveforms.

The sequence of at least three single, operator-controlled, independently programmed, narrow interval electrical waveforms, which have pulse intervals that are less than 100 milliseconds are referred to herein as “fast PulseAgile electrical waveforms” or as “narrow interval PulseAgile electrical waveforms”.

Preferably, the narrow interval electrical waveforms have a pulse interval of less than a few milliseconds.

With one embodiment of the method of the invention, step (a.) and step (b.) are carried out sequentially. For example, a DNA vaccine is first injected into the skin to form a bleb. Then, a needle electrode is placed on the skin at the bleb. In this respect, the “Derma Vax” system can be employed.

With another embodiment of the method of the invention, step (a.) and step (b.) are carried out simultaneously using an electrode that is pre-coated with the polynucleotide vaccine. In this respect, “Easy Vax” system can be employed.

In accordance with another aspect of the invention, an apparatus is provided for the delivery of polynucleotide vaccines to mammalian skin which includes a narrow interval electrical waveform generator, which is capable of applying a sequence of at least three single, operator-controlled, independently programmed, narrow interval electrical waveforms, which have pulse intervals that are less than 100 milliseconds; and which includes an electrode which is adapted to contact the skin into which a polynucleotide vaccine has been applied.

The sequence of at least three waveforms has one, two, or three of the following characteristics (1) at least two of the at least three waveforms differ from each other in waveform amplitude, (2) at least two of the at least three waveforms differ from each other in waveform width, and (3) a first waveform interval for a first set of two of the at least three waveforms is different from a second waveform interval for a second set of two of the at least three waveforms, and an electrode is connected to the narrow interval electrical waveform generator.

With one embodiment of the apparatus of the invention, the polynucleotide vaccine is applied to the skin prior to contacting the skin with the electrode. This can be accomplished by using a hypodermic needle.

With another embodiment of the apparatus of the invention, the polynucleotide vaccine is pre-coated on the electrode and is applied to the skin at the same time the electrode is contacted with the skin.

The apparatus that provides fast PulseAgile electrical waveforms or narrow interval PulseAgile electrical waveforms and that employs any suitable electrode for application to mammalian skin is made by Cyto Pulse, Inc., 810 Cromwell Park Drive, Suite T, Glen Burnie, M D 21061, and is known by the name “Derma Vax”.

In this respect, a two page Data Sheet entitled “Derma Vax™ Clinical Evaluation Intra-dermal System” is disclosed by Cyto Pulse, Inc. on the Internet at the following address: http://www.cytopulse.com/Datasheet%20Derma%20Vax.pdf.

The two page Data Sheet entitled “Derma Vax™ Clinical Evaluation Intra-dermal System” is incorporated herein by reference.

The apparatus that provides fast PulseAgile electrical waveforms or narrow interval PulseAgile electrical waveforms and that employs a pre-coated electrode suitable for application to mammalian skin is also made by Cyto Pulse, Inc. and is known by the name “Easy Vax”.

The apparatus that provides fast PulseAgile electrical waveforms or narrow interval PulseAgile electrical waveforms is also made by Cyto Pulse, Inc. and is known by the name “CCEP-40 Waveform Generator”. As stated above, specifications for the “CCEP-40 Waveform Generator” are provided in the two page Data Sheet entitled “Derma Vax™ Clinical Evaluation Intra-dermal System”.

In accordance with another aspect of the invention, a method for the delivery of polynucleotide vaccines to mammalian skin is provided wherein a polynucleotide vaccine includes a polynucleotide which expresses an antigen from the CEA family of antigens.

In accordance with another aspect of the invention, a method for the delivery of polynucleotide vaccines to mammalian skin is provided wherein a polynucleotide vaccine includes a polynucleotide which expresses an antigen from an immunoglobulin family of antigens selected from the group consisting of antigens which include immunoglobulin superfamily antigens, such as Igs; TCRs; class I and II major histocompatibility complex (MHC) molecules; one-domain proteins of thymocytes and T-cells (Thy-1); myelin protein PO; beta 2-microglobulin; two-domain proteins—spnnge receptor tyrosine kinase (RTK), sponge adhesive protein (SAP), Drosophilia tyrosine-kinase receptor (DTKR), cortical-thymocyte receptors of Xenopus (CTX), human (CTH), etc.; and a large group of adhesins, coreceptors, and Ig receptors with varying number of domains.

In accordance with another aspect of the invention, a method for the delivery of polynucleotide vaccines to mammalian skin is provided wherein a polynucleotide vaccine includes a polynucleotide which expresses an antigen from human PSA or xenogenic PSA, wherein PSA means prostate specific antigen.

In accordance with another aspect of the invention, a method for the delivery of polynucleotide cancer vaccines to mammalian skin is provided which comprises the steps of:

(a.) administering a polynucleotide cancer vaccine into the skin at an administration site,

(b.) applying a needle electrode to the skin in the vicinity to the administration site,

(c.) applying a sequence of at least three single, operator-controlled, independently programmed, narrow interval electrical waveforms, which have pulse intervals that are less than 100 milliseconds, to deliver the polynucleotide cancer vaccine into skin cells by electroporation,

wherein the sequence of at least three waveforms has one, two, or three of the following characteristics: (1) at least two of the at least three waveforms differ from each other in waveform amplitude; (2) at least two of the at least three waveforms differ from each other in waveform width; and (3) a first waveform interval for a first set of two of the at least three waveforms is different from a second waveform interval for a second set of two of the at least three waveforms.

In accordance with another aspect of the invention, a method for the delivery of polynucleotide vaccines to mammalian skin is provided wherein said polynucleotide cancer vaccines are against cancers which express CEA, including colorectal carcinoma, gastric carcinoma, lung adenocarcinoma, pancreatic carcinoma, breast carcinoma, ovarian carcinomas, gall and urinary bladder carcinomas, endometrial adenocarcinoma, and small cell lung carcinoma.

The above brief description sets forth rather broadly the more important features of the present invention in order that the detailed description thereof that follows may be better understood, and in order that the present contributions to the art may be better appreciated. There are, of course, additional features of the invention that will be described hereinafter and which will be for the subject matter of the claims appended hereto.

In this respect, before explaining some implementations of the principles of the invention in greater detail below, it is understood that the invention is not limited in its application to the details of the construction and to the arrangements of the components set forth in the following description or illustrated in the drawings. The invention is capable of other embodiments and of being practiced and carried out in various ways. Also, it is to be understood, that the phraseology and terminology employed herein are for the purpose of description and should not be regarded as limiting.

As such, those skilled in the art will appreciate that the conception, upon which disclosure is based, may readily be utilized as a basis for designing other structures, methods, and systems for carrying out the several purposes of the present invention. It is important, therefore, that the claims be regarded as including such equivalent constructions insofar as they do not depart from the spirit and scope of the present invention.

It is therefore an object of the present invention is to provide a new and improved method and apparatus for the delivery of polynucleotide vaccines to mammalian skin which provides a method and apparatus for the delivery of polynucleotide vaccine to mammalian skin which takes less than 3.5 seconds to administer the polynucleotide vaccine.

Still another object of the present invention is to provide a new and improved method and apparatus for the delivery of polynucleotide vaccines to mammalian skin that applies plural PulseAgile electrical waveforms to the mammalian skin and only causes one muscle contraction for the plural applied electrical waveforms.

Yet another object of the present invention is to provide a new and improved method and apparatus for the delivery of polynucleotide vaccines to mammalian skin which gives evidence of successful genetic expression of the administered polynucleotide vaccine.

Even another object of the present invention is to provide a new and improved method and apparatus for the delivery of polynucleotide vaccines to mammalian skin that gives evidence of providing a desired protein which results from the successful genetic expression of the polynucleotide vaccine.

Even another object of the present invention is to provide a new and improved method and apparatus for the delivery of polynucleotide vaccines wherein the polynucleotide vaccine can include polynucleotides that are expressed as antigens which include carcinoembryonic antigen (CEA) and the human CEA family which comprises 29 genes of which 18 are expressed, wherein 7 belong to the CEA subgroup and 11 to the pregnancy specific glycoprotein subgroup.

Still another object of the present invention is to provide a new and improved method and apparatus for the delivery of polynucleotide vaccines wherein the polynucleotide vaccine can include polynucleotides that are expressed as antigens which include immunoglobulin superfamily antigens, such as Igs; TCRs; class I and II major histocompatibility complex (MHC) molecules; one-domain proteins of thymocytes and T-cells (Thy-1); myelin protein PO; beta 2-microglobulin; two-domain proteins—spnnge receptor tyrosine kinase (RTK), sponge adhesive protein (SAP), Drosophilia tyrosine-kinase receptor (DTKR), cortical-thymocyte receptors of Xenopus (CTX), human (CTH), etc.; and a large group of adhesins, coreceptors, and Ig receptors with varying number of domains.

Yet another object of the invention is to provide a method and apparatus for the delivery of polynucleotide vaccines against cancers which express CEA, including colorectal carcinoma, gastric carcinoma, lung adenocarcinoma, pancreatic carcinoma, breast carcinoma, ovarian carcinomas, gall and urinary bladder carcinomas, endometrial adenocarcinoma, and small cell lung carcinoma.

These together with still other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

These together with still other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF DRAWINGS

The invention will be better understood and the above objects as well as objects other than those set forth above will become more apparent after a study of the following detailed description thereof. Such description makes reference to the annexed drawing wherein:

FIG. 1 is a graph illustrating a comparison of luciferase expression resulting from the application of fast PulseAgile electrical waveforms versus the application of slow PulseAgile electrical waveforms for delivery of luciferase plasmid with electroporation using “Derma Vax” equipment.

FIG. 2 a graph illustrating a comparison of T cell response to vaccination using Dengue 1 DNA vaccine, resulting from the application of fast PulseAgile electrical waveforms versus the application of slow PulseAgile electrical waveforms for delivery of the vaccine with electroporation using “Derma Vax” equipment.

FIG. 3 is a graph illustrating a comparison of luciferase expression resulting from the application of fast PulseAgile electrical waveforms versus the application of slow PulseAgile electrical waveforms for delivery of luciferase plasmid with electroporation using “Derma Vax” equipment.

FIG. 4 shows two side-by-side graphs illustrating CEA-specific T cell responses after 2× tet-wtCEA immunization. BALB/c mice (n=7 per group) received 40 μg of tet-wtCEA DNA by intradermal needle injection+/−fast Pulse Agile Dermavax electroporation, at two time points 4 weeks apart. IFN-gamma ELISpot readout against CD8− or CD4 restricted CEA peptides.

FIG. 5 shows induction of immune responses to CEA after a single immunization with tet-wtCEA DNA in combination with Fast PulseAgile™ and Derma Vax™ electroporation. More specifically, in FIG. 5, CD4 and CD8 T cell responses are shown after a single immunization with tet-wtCEA DNA+electroporation. Two weeks post immunization, CEA-specific immune responses were analyzed by IFN-gamma ELISpot using CD8 or CD4 restricted CEA peptides.

FIG. 6 shows induction of immune responses to CEA after a single immunization with tet-wtCEA DNA in combination with Fast PulseAgile™ and Derma Vax™ electroporation. More specifically, in FIG. 6, CEA-specific antibody responses are shown after immunization with tet-wtCEA DNA+electroporation. Two weeks after DNA immunization ELISA was used to analyze CEA-specific antibody responses.

### MODES FOR CARRYING OUT THE INVENTION

A method and apparatus are provided for the delivery of polynucleotide cancer vaccines to mammalian skin, and with reference to the drawings, said method and apparatus are described below.

Specifications for “Derma Vax” and “CCEP-40 Waveform Generator” are as follows:

DERMA VAX Specifications

```
Operation
    Mode 1 - Vaccine Delivery by trained health
professional
        Touch Screen
            Opening Screen for parameter entry
                Patient ID entry
                Vaccine ID entry
                Electrode ID entry
            Vaccination Screen
                SKIN - measure skin resistance every
                            second and display
                READY - turn on high voltage power
                                supply
                START - start pulsing
                DONE - vaccination completed
Mode 2 - Setup by trained IT specialist
        Pulse parameters
        Download data files
```

Delivery Electrode

| | | |
|---|---|---|
| Vaccine delivery volume | 2 blebs × 25 μl each | IDA-4-6 |
| | 2 blebs × 50 μl each | IDA-6-6 |
| Delivery target | skin/dermis | |
| Electrode | | |

| | |
|---|---|
| Handle | Reusable with alcohol cleaning |
| Tip | Sterile |
| | Single packaged |
| | Disposable |

| | IDA-4-4 | | IDA-4-6 | | IDA-6-6 | |
|---|---|---|---|---|---|---|
| Row spacing | 4 | mm | 4 | mm | 6 | mm |
| Needles/row | 4 | | 6 | | 6 | |
| Needle spacing | 1.5 | mm | 1.5 | mm | 1.5 | mm |
| Needle | 0.3 | mm | 0.3 | mm | 0.3 | mm |
| diameter | | | | | | |
| Needle length | 2 | mm | 3 | mm | 3 | mm |
| V/d maximum | 2500 | v/cm | 2500 | v/cm | 1667 | v/cm |

| CCEP-40 Waveform Generator | | |
|---|---|---|
| Pulsing | | |
| Skin resistance pulsing | 4 μs at 5 volts every | |
| | second | |
| Pulse Protocol Parameters | | |
| Parameters in a Group | | |
| Pulse Width | 50 μs to 1 ms | 50 to 1000 volts |
| | 50 μs to 10 ms | 50 to 300 volts |

| | |
|---|---|
| Pulse current trip | 26 amps |
| Load Range | 15 to 1500 ohms |
| Number of pulses | 1 to 10 |
| Maximum Duty Cycle | 50% |
| Interval | 200 μs to 1 sec (pulse |
| | start to pulse start) |
| Number of Groups | 3 |
| Pulse Measurement | |
| Internal Digitizer | |
| Levels | 12 bit |
| Samples | Pulse width/8 minimum 100 μs |
| Data stored internally and on | |
| external USB Key | |
| Data Types | |
| Raw data: DV<Date>.xml | |
| Log Data DV<Date>.txt | |
| CSV Data DV<Date>.csv | |

All data automatically stored in internal memory and
may be downloaded to an external USB Key
Maximum Data Logs stored and retrievable from
internal flash memory >20,000
Front Panel

| | |
|---|---|
| Computer | |
| Operating System | Windows ™ Mobile 6.0 |
| Interface | Touch screen |

Line/Mains Switch with illumination
Emergency Stop Button (resets computer to ready state)
Touch Screen

| | |
|---|---|
| USB Ports | 2 |
| Electrode connector | Fiscer Series 4032 |
| Back Panel | |
| Power Entry | IEC 320 |
| Ethernet | RJ45 |
| Electrical and Mechanical | |
| CCEP-40A Cabinet with handle | 32 mm w × 20 mm h × 40 mm l |
| | 12.6 in w × 7.9 in h × |
| | 15.7 in l |
| Weight | 25 pounds, 11.3 kg |
| Operating temperature | 10 to 40° C. |
| Mains Voltage | 100 to 250 vac |
| Fuse | 5 A slo blo, 5 mm × 20 mm |
| Power reserve | >5 minutes after power |
| | fail |

Experiments for carrying out the method of the invention employing apparatus of the invention for the delivery of polynucleotide vaccines to mammalian skin are set forth below.

### Experiment 1

Purpose and Scope

The purpose of this experiment is to compare fast PulseAgile electrical waveforms (using the Cyto Pulse “Derma Vax” system) versus slow PulseAgile electrical waveforms (using the Cyto Pulse PA-4000 system). The new Derma Vax system can deliver pulses more rapidly than the PA-4000.

Background

Dr. Anna-Karin Roos published at least two waveforms that induced good luciferase expression in the skin of mice. The system used was the PA-4000, and slow PulseAgile electrical waveforms were employed. New capabilities have been engineered into the Derma Vax system which employs the “CCEP-40 Waveform Generator”. One significant difference is that the Derma Vax system can deliver pulses with shorter pulse intervals. That is, with the “Derma Vax” system, pulse intervals of less than 100 milliseconds can be provided. This experiment will evaluate the effect on in vivo luciferase expression using fast PulseAgile electrical waveforms.

Approach

Plasmid used: gWizLuciferase from Aldeveron at 5 mg/ml diluted to 0.5 mg/ml in sterile PBS.

### System: Derma Vax #F2LQ2608851

Electrode: Intradermal Array (4 mm gap, 6 needles per row, 2 rows) parallel row electrode.
Injections: Mice were restrained using a 50 ml conical tube modified with breathing holes. The mouse was inserted head first into the tube. The tail was draped over my left index finger. A small patch of hair was removed on the base of the tail using small scissors. Using a 27 gauge, 0.5 in needle on a tuberculin syringe, a 20 microliter intradermal injection was made on the right side of the base of the tail and sacrum. The site was marked using a Sharpee pen. The rows of needles were inserted around the injection site with the electrode gap oriented left to right and therefore the rows were aligned cranially and caudally. The selected electroporation protocol was initiated and the needles removed. This process was repeated on the left side of the sacrum.

Groups (shown as cages in results). All times are shown in milliseconds

| Protocols | Cage 1 | Cage 2 |
|---|---|---|
| V/d 1 | 1125 | 1125 |
| V1 | 450 | 450 |
| PW 1 | 0.05 | 0.05 |
| #1 | 1 | 1 |
| PI1 | 300 | 0.2 |
| V/d2 | 1125 | 1125 |
| V2 | 450 | 450 |
| PW 2 | 0.05 | 0.05 |
| # 2 | 1 | 1 |
| PI2 | 500 | 100 |
| V/d 3 | 275 | 275 |
| V3 | 110 | 110 |
| PW 3 | 10 | 10 |
| #3 | 8 | 8 |
| PI 3 | 300 | 20 |

Mice were returned to their cages.

After 18-24 hours, the mice were euthanized using CO2 inhalation. Tissue from each of the two sites was incised using a 6 mm punch biopsy. Subcutaneous tissue was removed suing scissors and the skin with subcutaneous tissue was added to 1 ml of lysis buffer. The sample was kept on ice until the assay.

Tissues were homogenized using a model IKA tissue homogenizer. A 50 microliter sample of the 1 ml homogenate was added to a white assay plate. Standards were made by diluting a know amount of luciferase with lysis buffer using a three fold dilution series. 50 microliter reagent A of the luciferase assay kit was added to each well. The plate was added to the 96 well luminometer. 50 microliter of reagent B was added and the resulting light was measured over one second.

Data was exported to an Excel spreadsheet for data analysis.

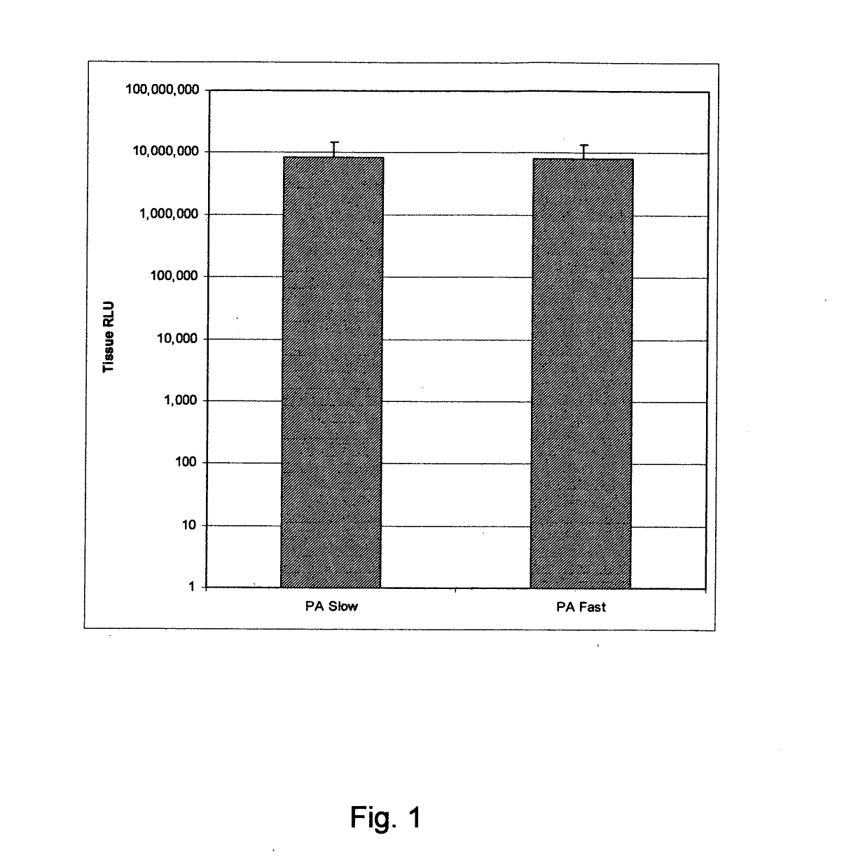

Reference is made to FIG. 1 in the drawings for a graphical representation of the results. There is a statistical equivalence of genetic expression between fast and slow electrical waveform protocols.

### Results

| | | ng/site |
|---|---|---|
| | Cage 1 | Cage 2 |
| | PA Slow | PA Fast |
| | 22 | 43 |
| | 464 | 510 |
| | 486 | 283 |
| | 180 | 267 |
| | 197 | 168 |
| Mean | 270 | 254 |
| SD | 200 | 172 |
| CV | 74 | 68 |

It is a surprising and unexpected result that electroporation of a polynucleotide vaccine into mammalian skin cells along with successful gene expression can occur with fast PulseAgile electrical waveforms having a pulse interval of less than 100 milliseconds.

It is an even greater surprising and unexpected result that electroporation of a polynucleotide vaccine into mammalian skin cells along with successful gene expression can occur with fast PulseAgile electrical waveforms having a pulse interval of a few milliseconds. Conventionally, it would be expected that the time constant of pulse intervals of only a few milliseconds would be too low for successful electroporation.

### Experiment 2

Purpose and Scope

The purpose of this experiment is to compare T cell responses induced by DNA immunization using fast PulseAgile electrical waveforms (using the Cyto Pulse “Derma Vax” system) versus slow PulseAgile electrical waveforms (using the Cyto Pulse PA-4000 system). The new Derma Vax system can deliver pulses more rapidly than the PA-4000. More specifically, the purpose of this study is to compare T cell responses induced by DNA immunization using Pulse Agile Derma Vax delivery with Dengue 1 plasmids expressing prM-E and NS1—NS3.

Background

Dr. Anna-Karin Roos published at least two waveforms that induced good luciferase expression in the skin of mice. The system used was the PA-4000, and slow PulseAgile electrical waveforms were employed. New capabilities have been engineered into the Derma Vax system which employs the “CCEP-40 Waveform Generator”.

One significant difference is that the Derma Vax system can deliver pulses with shorter pulse intervals. That is, with the “Derma Vax” system, pulse intervals of less than 100 milliseconds can be provided. This experiment will evaluate the effect on in vivo T cell responses using fast PulseAgile electrical waveforms.

Approach

Plasmid used: Dengue 1 prM-E and Dengue 1 NS1—NS3 at 5 mg/ml each diluted to 0.5 mg/ml in the same sterile PBS.

### System: Derma Vax #07-0215DV

Electrode: Intradermal Array (4 mm gap, 6 needles per row, 2 rows) parallel row electrode.
Injections: Mice were restrained using a 50 ml conical tube modified with breathing holes. The mouse was inserted head first into the tube. The tail was draped over my left index finger. A small patch of hair was removed on the base of the tail using small scissors. Using a 27 gauge, 0.5 in needle on a tuberculin syringe, a 20 μl intradermal injection was made on the right side of the base of the tail and sacrum. The rows of needles were inserted around the injection site with the electrode gap oriented left to right and therefore the rows were aligned cranially and caudally. The selected electroporation protocol was initiated and the needles removed. This process was repeated on the left side of the sacrum

Groups (shown as cages in results). All times are shown in milliseconds

| Protocols | Group P | Group O | Control |
|---|---|---|---|
| V/d 1 | 1125 | 1125 | 0 |
| V1 | 450 | 450 | 0 |
| PW 1 | 0.05 | 0.05 | 0 |
| # 1 | 1 | 1 | 0 |
| PI 1 | 0.2 | 300 | 0 |
| V/d 2 | 1125 | 1125 | |
| V2 | 450 | 450 | |
| PW 2 | 0.05 | 0.05 | |
| # 2 | 1 | 1 | |
| PI 2 | 30 | 300 | |
| V/d 3 | 275 | 275 | |
| 3 | 110 | 110 | |
| PW 3 | 10 | 10 | |
| # 3 | 8 | 8 | |
| PI 3 | 20 | 100 | |

Mice were returned to their cages.
At 2 weeks after immunization, mice were euthanized using CO2 inhalation and the spleens were collected for intracellular cytokine assay.

Results

Results show below are percent of CD8 positive cells that are gamma interferon positive.

Results are shown with background from un-immunized animals subtracted.

| | Fast (Group P) | Slow (Group O) |
|---|---|---|
| | 6.42 | 4.03 |
| | 5.38 | 6.11 |
| | 4.35 | 3.36 |
| | 4.28 | 2.75 |
| | 2.06 | 2.16 |
| Mean | 4.50 | 3.68 |
| SD | 1.62 | 1.53 |

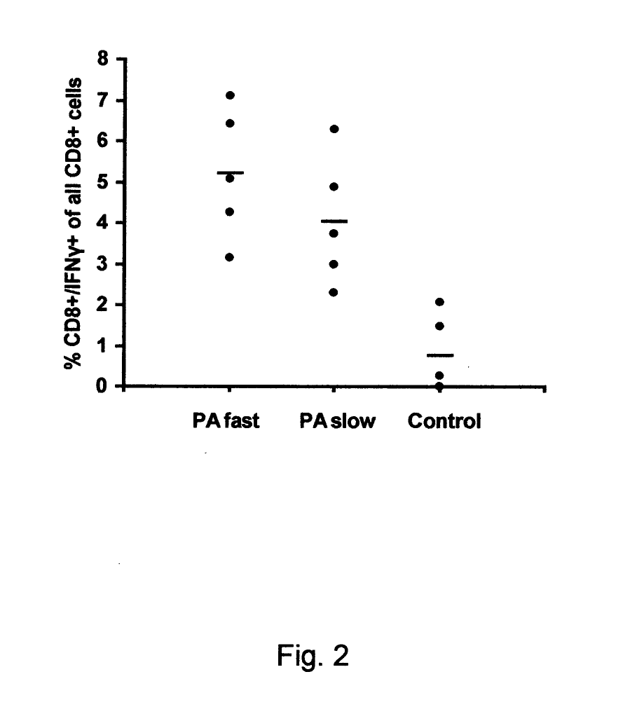

Reference is made to FIG. 2 in the drawings for a graphical representation of the test results. In this respect, by conducting a Student's T test, the test results show a statistically insignificant difference between fast and slow electrical waveform protocols. In this respect, there is a statistical equivalence of T cell enhancement between fast and slow electrical waveform protocols.

Conclusions

T cell responses induced by fast PulseAgile electrical waveforms with the “Derma Vax” system are equivalent to those induced by slow PulseAgile electrical waveforms with the “Derma Vax” system with in vivo electroporation.

TABLE I
Perceptible muscle contractions are reduced by electroporation using
fast PulseAgile electrical waveforms in contrast with slow PulseAgile
electrical waveforms.

| | | Slow Pulse |
|---|---|---|
| Parameter | Fast Pulse Agile | Agile |
| Pulses Delivered | 10 | 10 |
| Total Delivery Time | 0.23 Seconds | 3.5 Seconds |
| Perceptible Muscle | 1 | 10 |
| Contractions | | |

Clearly, with fast PulseAgile electrical waveforms (as compared with slow PulseAgile electrical waveforms), delivery time is much less than 3.5 seconds, and only 1 muscle contraction is perceived, even when 10 pulses are delivered.

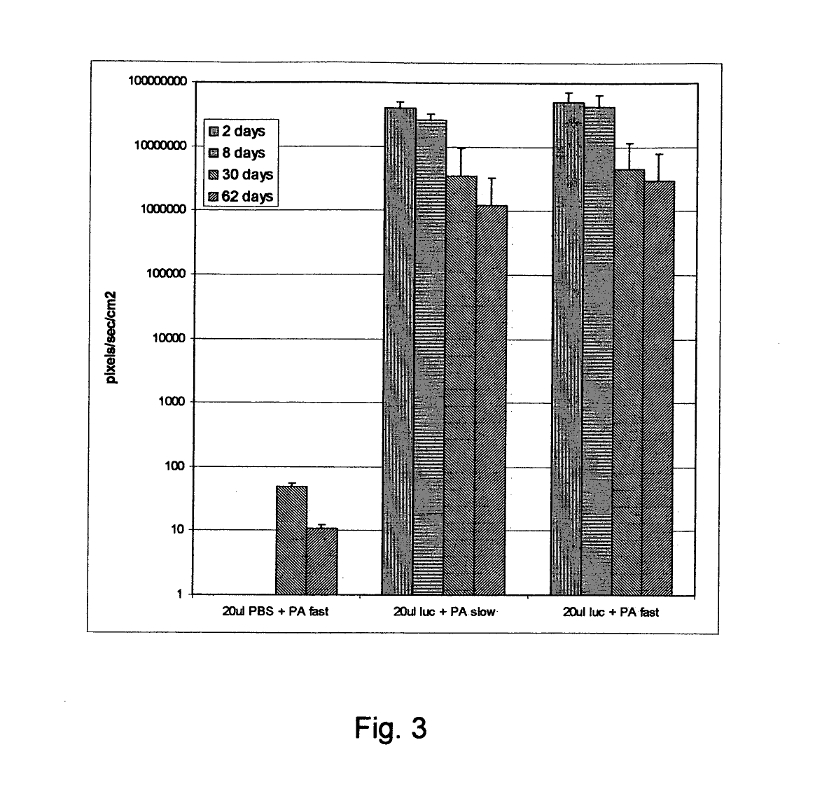

With respect to FIG. 3, DNA delivery (DNA being a polynucleotide) was carried out as follows.

Mice were anesthetized with 4% isoflurane (Baxter Medical AB, Kista, Sweden) and maintained at 2-2.5% isoflurane in a mask during immunizations. 20 μg DNA in PBS was injected intradermally on each flank, near the base of the tail, using a 29 G insulin grade syringe (Micro-Fine U-100, BD Consumer Healthcare, Franklin Lakes, N.J.).

Subsequently, a needle array electrode was placed over the raised skin area of injection and voltage was applied (2 pulses, en11125 V/cm, 50 μsec+8 pulses, 275 V/cm, 10 msec). Pulse intervals were varied to make fast and slow PulseAgile protocols.

The needle array electrode used was the Cyto Pulse Intradermal array (four needle, 4 mm gap, two rows) (Cyto Pulse Sciences, Inc. Glen Burnie, M D). Electroporation was performed using the Derma Vax Electroporation System (Cyto Pulse Sciences, Inc.).

### Experiment 3

Description of the Vaccine Plasmid

A. pKCMVtet-wtCEA

Vector frame; pKCMV, is a plasmid with 3555 bp containing multiple cloning sites. The plasmid gives E. coli resistance to kanamycin.

Promoter; Major immediate early promoter (MIE) from human cytomegalovirus is a strong eukaryotic promoter used frequently in eukaryotic systems (accession number K03104).

Gene; Modified Human Carcinoembryonic Antigen (CEA) (accession no M17303). A T-helper epitope (51 bp) from Tetanus toxoid (tet) have been cloned in between the 5′ signal sequence and the CEA sequence. Total size of tet+wtCEA is 2160 bp.

Termination sequence; Polyadenylation sequences from human papillomavirus type 16 (accession number K02718). This sequence is often used in eukaryotic expression systems.

Antibiotic resistance gene; Aminoglycoside 3′ phosphotransferase II accession number E02455), which gives kanamycin resistance from the Tn5 transposon.

| Nt position | Function |
|---|---|
| 1-746 | MIE promoter (nt 67-812 from K03104), modified. |
| 747-802 | Polylinker. |
| 803-2962 | tet-wtCEA |
| 2963-3026 | HPV 16 poly-A sequence. |
| 3027-5717 | pKCMV sequences: |
| 3331-4224 | E. coli Replication origin. |
| 4318-5111 | aminoglycoside 3′-phosphotransferase II (APH). |

### B. Modifications of the Plasmid Gene Insert.

Further modifications of the plasmid gene insert may include:
B1. Amino acid alterations to enhance epitope presentation.

Amino acid alterations to decrease glycosylation of the CEA protein product.

Modifications of the gene insert to include fragments of the CEA gene (e.g. the A3 & B3 regions)

B2. Alterations to increase breadth of immunity by including heterogeneous members of the CEACAM protein family; CEACAM1 (CD66a, BGP), CEACAM6 (CD66c, NCA), and homologous genes of non-human origin.

### B3. Combinations of B1 and B2.

DNA Injections and In Vivo Electroporation

Intradermal injections with 10-40 micrograms DNA/20 microliters PBS were made on the lower back of a mouse, near the base of the tail, using an 29 G insulin grade syringe (Micro-Fine U-100, BD Consumer Healthcare, Franklin Lakes, N.J.). Immediately after intradermal DNA administration, a needle array electrode was placed over the raised skin area of injection and voltage was applied (2 pulses, 1125 V/cm, 50 μsec+8 pulses, 275 V/cm, 10 msec). The number, amplitude and length of pulses were always the same, only the interval between the pulses varied (described in FIG. 1A). Needle array electrodes consisted of two parallel rows of four or six 2-mm pins (1.5×4 mm gaps) (Cyto Pulse Sciences, Inc. Glen Burnie, Md.). Electroporation was performed using the PA-4000S—Advanced PulseAgile® Rectangular Wave Electroporation System and software or the DERMA VAX™ Clinical DNA vaccine delivery system (both from Cyto Pulse Sciences, Inc.).

| | Slow | Fast |
|---|---|---|
| Protocols | PulseAgile | PulseAgile |
| V/d 1 | 1125 | 1125 |
| V1 | 450 | 450 |
| PW 1 | 0.05 | 0.05 |
| #1 | 1 | 1 |
| PI1 | 300 | 0.2 |
| V/d2 | 1125 | 1125 |
| V2 | 450 | 450 |
| PW 2 | 0.05 | 0.05 |
| # 2 | 1 | 1 |
| PI2 | 500 | 100 |
| V/d 3 | 275 | 275 |
| V3 | 110 | 110 |
| PW 3 | 10 | 10 |
| #3 | 8 | 8 |
| PI 3 | 300 | 20 |

Results

Conclusion: Intradermal Delivery of CEA DNA Vaccines in Combination with Derma Vax™ Fast PulseAgile™ Electroporation Results in Increased Immune Responses to CEA.

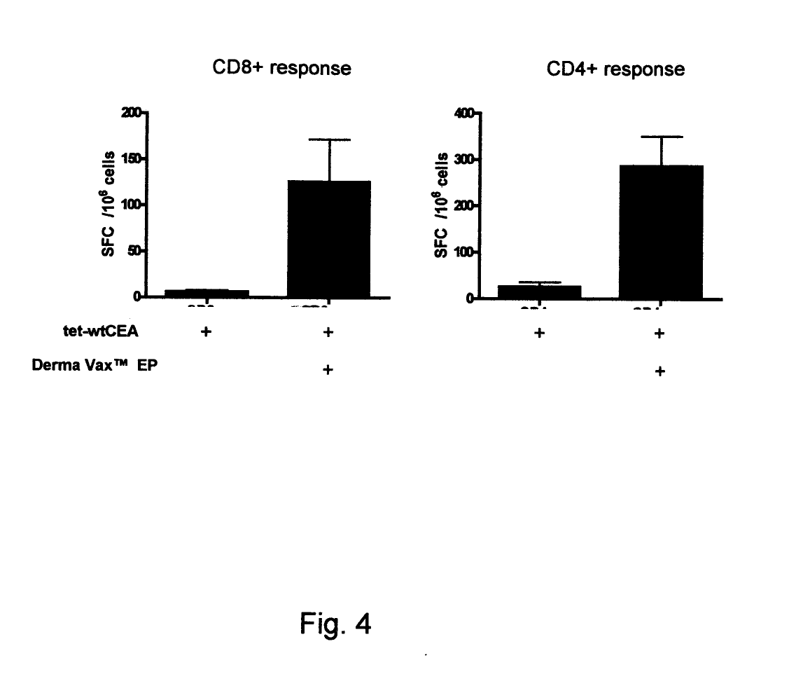

To investigate the effect of Derma Vax™ electroporation on the induction of CEA-specific immune responses after DNA vaccination, BALB/c mice were immunized twice with intradermal injections of tet-wtCEA DNA four weeks apart in combination with Derma Vax™ Fast PulseAgile™ electroporation (control animals received intradermal injection of tet-wtCEA DNA but no electroporation). Ten days after the last immunization, CEA-specific T cell responses were analyzed by standard IFN-gamma ELISpot. As shown in FIG. 4, both CD8+ and CD4+ T cell responses were markedly increased after electroporation. In contrast, in animals receiving intradermal injections of tet-wtCEA DNA without electroporation, no CEA-specific immune response was detectable.

Stated somewhat differently, as disclosed above with respect to the description of the drawings, FIG. 4 shows two side-by-side graphs illustrating CEA-specific T cell responses after 2× tet-wtCEA immunization. BALB/c mice (n=7 per group) received 40 μg of tet-wtCEA DNA intradermal needle injection+/−Fast PulseAgile Derma Vax electroporation, at two time points 4 weeks apart. IFN-gamma ELISpot readout against CD8− or CD4 restricted CEA peptides.

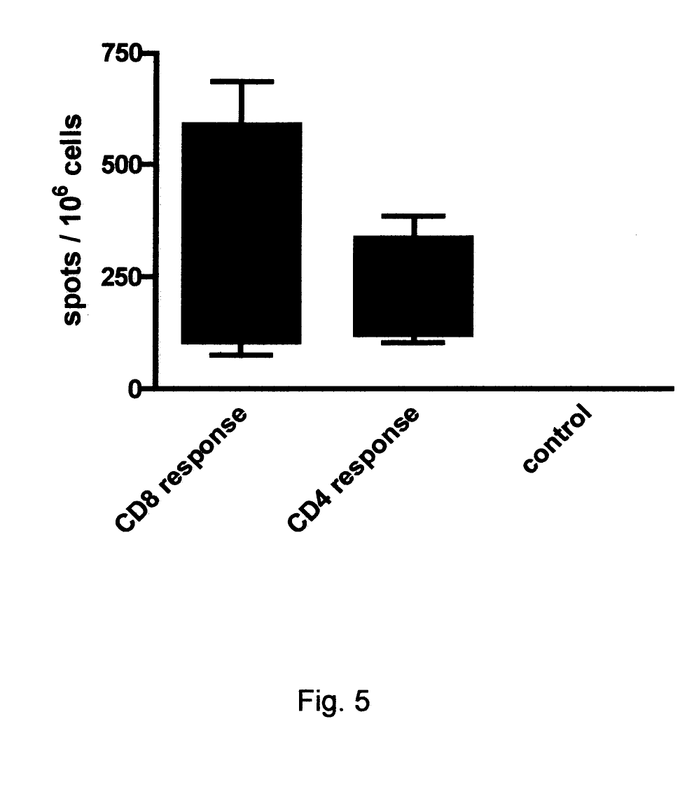

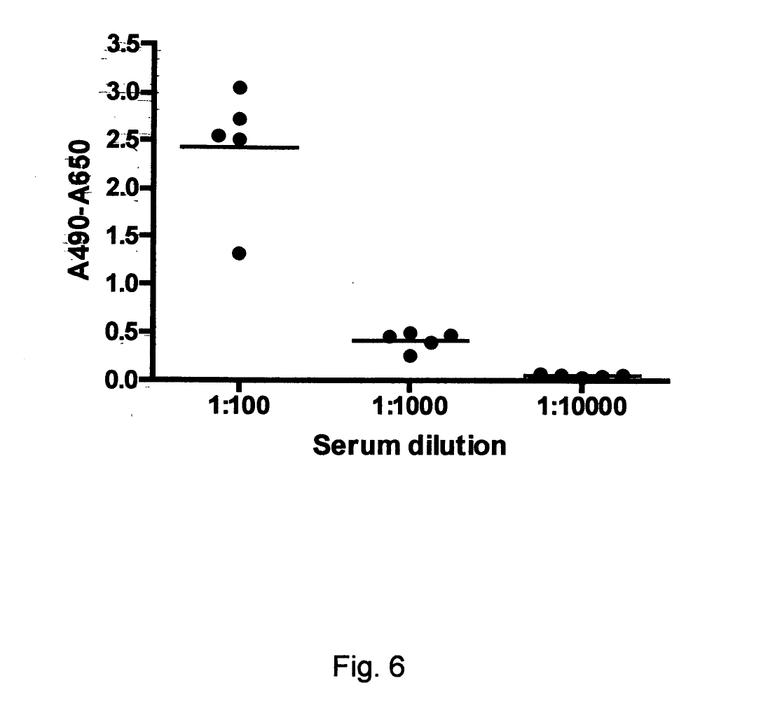

In a follow-up experiment, assessments were made with respect to the immune responses to CEA after a single immunization of BALE/c mice with tet-wtCEA DNA followed by electroporation using the Fast PulseAgile electroporation protocol. Two weeks post-immunization, mouse splenocytes and serum were collected for analysis of CEA specific immune responses. As shown in FIG. 5, a single immunization with tet-wtCEA DNA in combination with electroporation induced high CD8+ (more than 500 spots/10⁶ cells) and CD4+ T cell responses. Moreover, serum analysis by ELISA revealed that a single immunization was enough to stimulate an antibody response to CEA (FIG. 6).

Stated somewhat differently, as disclosed above with respect to the description of the drawings, FIG. 5 shows induction of immune responses to CEA after a single immunization with tet-wtCEA DNA in combination with Fast PulseAgile™ and Derma Vax™ electroporation. More specifically, in FIG. 5, CD4 and CD8 T cell responses are shown after a single immunization with tet-wtCEA DNA+electroporation. Two weeks post immunization, CEA-specific immune responses were analyzed by IFN-gamma ELISpot using CD8 or CD4 restricted CEA peptides.

Also, stated somewhat differently, as disclosed above with respect to the description of the drawings, FIG. 6 shows induction of immune responses to CEA after a single immunization with tet-wtCEA DNA in combination with Fast PulseAgile™ and Derma Vax™ electroporation. More specifically, in FIG. 6, CEA-specific antibody responses are shown after immunization with tet-wtCEA DNA+electroporation. Two weeks after DNA immunization ELISA was used to analyze CEA-specific antibody responses.

In view of the above, it is apparent that the present invention accomplishes all of the objects set forth by providing a new and improved method and apparatus for the delivery of polynucleotide vaccines to mammalian skin that may advantageously be used to which takes less than 3.5 seconds to administer the polynucleotide vaccine. With the invention, a method and apparatus for the delivery of polynucleotide vaccines to mammalian skin are provided which applies plural PulseAgile electrical waveforms to the mammalian skin and only causes one muscle contraction for the plural applied electrical waveforms. With the invention, a method and apparatus for the delivery of polynucleotide vaccines to mammalian skin is provided which gives evidence of successful genetic expression of the administered polynucleotide vaccine. With the invention, a method and apparatus for the delivery of polynucleotide vaccines to mammalian skin are provided which gives evidence of providing a desired protein which results from the successful genetic expression of the polynucleotide vaccine.

As to the manner of usage and operation of the instant invention, the same is apparent from the above disclosure, and accordingly, no further discussion relative to the manner of usage and operation need be provided.

Thus, while the present invention has been shown in the drawings and fully described above with particularity and detail in connection with what is presently deemed to be the most practical and preferred embodiment(s) of the invention, it will be apparent to those of ordinary skill in the art that many modifications thereof may be made without departing from the principles and concepts set forth herein, including, but not limited to, variations in size, materials, shape, form, function and manner of operation, assembly and use.

What is claimed is:

1. A method for the delivery of polynucleotide vaccines to mammalian skin, comprising the steps of:
(a.) administering a polynucleotide vaccine into the skin at an administration site,
(b.) applying a needle electrode to the skin in the vicinity to the administration site, and
(c.) applying a sequence of at least three single, operator-controlled, independently programmed, narrow interval electrical waveforms, which have pulse intervals that are less than 100 milliseconds, to deliver the polynucleotide vaccine into skin cells by electroporation,
wherein the sequence of at least three waveforms has one, two, or three of the following characteristics: (1) at least two of the at least three waveforms differ from each other in waveform amplitude; (2) at least two of the at least three waveforms differ from each other in waveform width; and (3) a first waveform interval for a first set of two of the at least three waveforms is different from a second waveform interval for a second set of two of the at least three waveforms, and
wherein a polynucleotide vaccine includes a polynucleotide which expresses an antigen selected from the group consisting of immunoglobulin superfamily antigens.

2. The method of claim 1 wherein step (a.) and step (b.) are carried out sequentially.

3. The method of claim 1 wherein step (a.) and step (b.) are carried out simultaneously using an electrode that is pre-coated with the polynucleotide vaccine.

4. The method of claim 1 wherein the polynucleotide vaccine includes a polynucleotide which expresses an antigen selected from the group consisting of the CEA family of antigens.

5. The method of claim 1 wherein the polynucleotide vaccine includes a polynucleotide which expresses an immunoglobulin superfamily antigen selected from the group consisting of Igs; TCRs; class I and II major histocompatibility complex (MHC) molecules; one-domain proteins of thymocytes and T-cells (Thy-1); myelin protein PO; beta 2-microglobulin; two-domain proteins—spnnge receptor tyrosine kinase (RTK), sponge adhesive protein (SAP), Drosophilia tyrosine-kinase receptor (DTKR), cortical-thymocyte receptors of Xenopus (CTX), human (CTH), etc.; and a large group of adhesins, coreceptors, and Ig receptors with varying number of domains.

6. A method for the delivery of polynucleotide cancer vaccines to mammalian skin, comprising the steps of:
(a.) administering a polynucleotide cancer vaccine into the skin at an administration site,
(b.) applying a needle electrode to the skin in the vicinity to the administration site, and
(c.) applying a sequence of at least three single, operator-controlled, independently programmed, narrow interval electrical waveforms, which have pulse intervals that are less than 100 milliseconds, to deliver the polynucleotide cancer vaccine into skin cells by electroporation,
wherein the sequence of at least three waveforms has one, two, or three of the following characteristics: (1) at least two of the at least three waveforms differ from each other in waveform amplitude; (2) at least two of the at least three waveforms differ from each other in waveform width; and (3) a first waveform interval for a first set of two of the at least three waveforms is different from a second waveform interval for a second set of two of the at least three waveforms, and
wherein said polynucleotide cancer vaccines are against cancers which express CEA, including colorectal carcinoma, gastric carcinoma, lung adenocarcinoma, pancreatic carcinoma, breast carcinoma, ovarian carcinomas, gall and urinary bladder carcinomas, endometrial adenocarcinoma, and small cell lung carcinoma.
